(19) United States
(12) Patent Application Publication
(10) Pub. No.: US 2019/0015303 A1
(43) Pub. Date: Jan. 17, 2019

(54) COSMETIC PRODUCT HAVING IMPREGNATION MEMBER EMBOSSED AND ENGRAVED BY MELTING SURFACE

(71) Applicant: AMOREPACIFIC CORPORATION, Seoul (KR)

(72) Inventors: Sang Young JUNG, Seoul (KR); Hyeong Sam KIM, Seoul (KR); Jae Young Choi, Seoul (KR)

(21) Appl. No.: 15/557,433

(22) PCT Filed: Feb. 15, 2016
(86) PCT No.: PCT/KR2016/001476

§ 371 Date: Sep. 11, 2017

(30) Foreign Application Priority Data
Mar. 23, 2015 (KR) 10-2015-0040328

(51) Int. Cl. A61K 8/02; A45D 40/22; B29C 59/02

(52) U.S. Cl. CPC A61K 8/0208; A45D 33/008; B29C 59/022; A45D 40/22

(57) ABSTRACT

The present invention relates to a cosmetic product having an impregnation member embossed and engraved by melting the surface thereof, wherein the impregnation member is embossed and engraved on the surface by compressing the surface of the impregnation member onto a heated embossed and engraved metal mold and then melting the surface, and the open cell structure of the impregnation member's surface is melted to convert the same into an open cell structure having a size of ½ or less, and thus, the impregnation member prevents the contents from being wasted by excessively smearing the contents more than necessary if the impregnation member is put with a puff for the use of the content, and the contents can be uniformly and thinly smeared on the puff to efficiently use the content.

### CROSS-REFERENCE TO RELATED APPLICATION

This application claims the benefit of Korean application No. 10-2015-0040328, filed on Mar. 23, 2015 with the Korean Intellectual Property Office, the disclosure of which is incorporated herein by reference.

### TECHNICAL FIELD

The present invention relates to a cosmetic product having an impregnation member embossed and engraved by melting a surface of the impregnation member. More specifically, the present invention relates to a cosmetic product having an impregnation member embossed and engraved by melting a surface of the impregnation member, wherein the surface of the impregnation member is embossed and engraved by compressing the surface of the impregnation member onto a heated metal mold formed with an emboss-engrave pattern and melting the surface, and the open cell structure of the surface of the impregnation member is melted to convert the same into an open cell structure having a size of ½ or less. Thus, when the impregnation member is put with a puff to use the contents, the impregnation member prevents the contents from being wasted by excessively smearing the contents more than necessary, and the contents can be uniformly and thinly smeared on the puff so as to be efficiently used.

Moreover, the present invention relates to a cosmetic product having an impregnation member embossed and engraved by melting a surface of the impregnation member, wherein the impregnation member is embossed and engraved only on an edge portion of the upper surface so as to control the amount of contents discharged through a central portion of the impregnation member and discharged through an edge portion of the impregnation member when the contents are discharged, so that the contents are prevented from flowing out of a cosmetic container.

### BACKGROUND ART

A cosmetic product refers to an article which is used for a human body in order to add the charm of the human body and change the appearance of the human body brighter by making the human body clean and beautiful, or to maintain or promote the health of the skin or hair, while exerting slight effects on the human body.

In general, the cosmetic product is manufactured by mixing mutually different cosmetic materials by using an emulsifying agent such as a surface active agent, and the cosmetic materials may be classified into a water-in-oil (W/O) emulsion type cosmetic material and an oil-in-water (O/W) emulsion type cosmetic material according to the bonding structure between a water-based material and an oil-based material.

The water-in-oil emulsion type cosmetic material, which is obtained by bonding the oil-based material to an outside of the water-based material, has a larger quantity of oil, so that the water-in-oil emulsion type cosmetic material is slowly absorbed into skin and the feeling is heavy upon use, whereas the persistence is longer than that of the oil-in-water emulsion type cosmetic material. Thus, the cosmetic product requiring persistence is manufactured by using the W/O emulsion type cosmetic material to increase water resistance against sweat and water.

In order to compensate the defects of the W/O emulsion type cosmetic material where the feeling is heavy and sticky upon use, the viscosity of content is reduced upon the manufacture of the cosmetic product. However, when the water-in-oil product having low viscosity is stored in a container for a long period of time during distribution, the aqueous material of internal phase and the oil material of external phase may be separated from each other. In this case, it is inconvenient for a user to shake the container to mix the separated aqueous and oil materials with each other for use.

To solve the problems described above, as shown in FIG. 1, a product disclosed in Korean Patent Registration No. 10-1159877 filed by the applicant of the present application has been developed, in which an impregnation member 1 impregnated with water-in-oil contents having low viscosity is mounted in a compact container.

However, according to the above-described related art, since the contents are fully filled in the impregnation member 1 when the cosmetic product is used for the first time, even when the pressure is applied with a small force, the contents are put on the puff more than necessary and wasted.

### DISCLOSURE

### Technical Problem

To solve the problems described above, an object of the present invention is to provide a cosmetic product having an impregnation member embossed and engraved by melting a surface of the impregnation member, wherein the surface of the impregnation member is embossed and engraved by compressing the surface of the impregnation member onto a heated metal mold formed with an emboss-engrave pattern and melting the surface, and the open cell structure of the surface of the impregnation member is melted to convert the same into an open cell structure having a size of ½ or less. Thus, when the impregnation member is put with a puff to use the contents, the impregnation member prevents the contents froa being wasted by excessively smearing the contents more than necessary, and the contents can be uniformly and thinly smeared on the puff so as to be efficiently used.

In addition, an object of the present invention is to provide a cosmetic product having an impregnation member embossed and engraved by melting a surface of the impregnation member, wherein the impregnation member is embossed on the surface in various sizes of a convex circular or polygonal shape, so that various designs can be implemented to broaden the range of a consumer's choice.

In addition, an object of the present invention is to provide a cosmetic product having an impregnation member embossed and engraved by melting a surface of the impregnation member, wherein since the open cell structure of the impregnation member can be partially occluded when the upper surface of the impregnation member is melted with heat so as to be formed with the emboss-engrave pattern, the surface of the impregnation member melted and formed with the emboss-engrave pattern is pressed with a plurality of needles to allow the occluded surface to be perforated, so that the contents impregnated in the impregnation member can be smoothly discharged.

Moreover, an object of the present invention is to provide a cosmetic product having an impregnation member embossed and engraved by melting a surface of the impregnation member, wherein the impregnation member is embossed and engraved only on an edge portion of the upper surface so as to control the amount of contents discharged through a central portion of the impregnation member and discharged through an edge portion of the impregnation member when the contents are discharged, so that the contents are prevented from flowing out of a cosmetic container.

### Technical Solution

According to the present invention, there is provided a cosmetic product having an impregnation member embossed and engraved by melting a surface of the impregnation member, the cosmetic product including:

an outer container (10) having an open top;

an outer container lid (20) coupled to one side of the outer container (10);

an inner container (30) mounted inside the outer container (10);

the impregnation member (40) mounted inside the inner container (30) and impregnated with a cosmetic material; and

an inner container lid (70) hinged to one side of the inner container (30) so as to be opened and closed,

wherein the surface (41) of the impregnation member (40) is embossed and engraved by inserting and compressing the impregnation member (40) in a heated metal mold (50) formed with an emboss-engrave pattern and melting the surface (41).

In addition, a fixing member (60) may be further coupled to an upper end of the inner container (30) to fix the impregnation member (40) such that the impregnation member (40) is prevented from being separated to an outside.

In addition, the surface (41) of the impregnation member (40) may be melted and occluded by the metal mold (50), and the occluded surface (41) may be perforated by a plurality of needles.

In addition, the impregnation member (40) may be embossed and engraved only on an edge portion of the surface (41).

Moreover, the impregnation member (40) may be embossed on the surface (41) in a convex circular or polygonal shape.

### Advantageous Effects

According to the cosmetic product having the impregnation member embossed and engraved by melting a surface of the impregnation member of the present invention, the surface of the impregnation member is embossed and engraved by compressing the surface of the impregnation member onto a heated metal mold formed with an emboss-engrave pattern and melting the surface, and the open cell structure of the surface of the impregnation member is melted to convert the same into an open cell structure having a size of ½ or less. Thus, when the impregnation member is put with a puff to use the contents, the impregnation member can prevent the contents from being wasted by excessively smearing the contents more than necessary, and the contents can be uniformly and thinly smeared on the puff so as to be efficiently used.

In addition, according to the cosmetic product having the impregnation member embossed and engraved by melting a surface of the impregnation member of the present invention, the impregnation member is embossed on the surface in various sizes of a convex circular or polygonal shape, so that various designs can be implemented to broaden the range of a consumer's choice.

In addition, according to the cosmetic product having the impregnation member embossed and engraved by melting a surface of the impregnation member of the present invention, since the open cell structure of the impregnation member can be partially occluded when the upper surface of the impregnation member is melted with heat so as to be formed with the emboss-engrave pattern, the surface of the impregnation member melted and formed with the emboss-engrave pattern is pressed with a plurality of needles to allow the occluded surface to be perforated, so that the contents impregnated in the impregnation member can be smoothly discharged.

Moreover, according to the cosmetic product having the impregnation member embossed and engraved by melting a surface of the impregnation member of the present invention, the impregnation member is embossed and engraved only on an edge portion of the upper surface so as to control the amount of contents discharged through a central portion of the impregnation member and discharged through an edge portion of the impregnation member when the contents are discharged, so that the contents can be prevented from flowing out of a cosmetic container.

### DESCRIPTION OF DRAWINGS

FIG. 1 is a perspective view showing a compact container according to the related art.

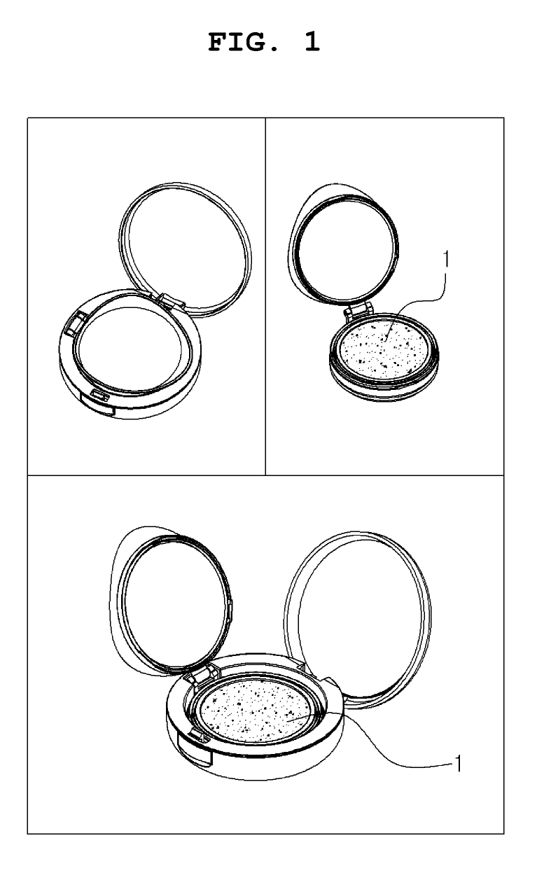

FIG. 2 is a perspective view showing a cosmetic product having an impregnation member embossed and engraved by melting a surface of the impregnation member according to an embodiment of the present invention.

FIG. 3 is an exploded perspective view showing a cosmetic product having an impregnation member embossed and engraved by melting a surface of the impregnation member according to an embodiment of the present invention.

FIG. 4 is a sectional view showing a cosmetic product having an impregnation member embossed and engraved by melting a surface of the impregnation member according to an embodiment of the present invention.

FIG. 5 is a perspective view showing a cosmetic product having an impregnation member embossed and engraved by melting a surface of the impregnation member according to another embodiment of the present invention.

FIG. 6 is a sectional view showing a cosmetic product having an impregnation member embossed and engraved by melting a surface of the impregnation member according to another embodiment of the present invention.

FIG. 7 is a sectional view showing a state of forming an impregnation member accommodated in a cosmetic product having the impregnation member embossed and engraved by melting a surface of the impregnation member according to an embodiment of the present invention.

### BEST MODE

[Mode for Invention]

Hereinafter, a cosmetic product having an impregnation member embossed and engraved by melting a surface of the impregnation member according to an embodiment of the present invention will be described with reference to accompanying drawings.

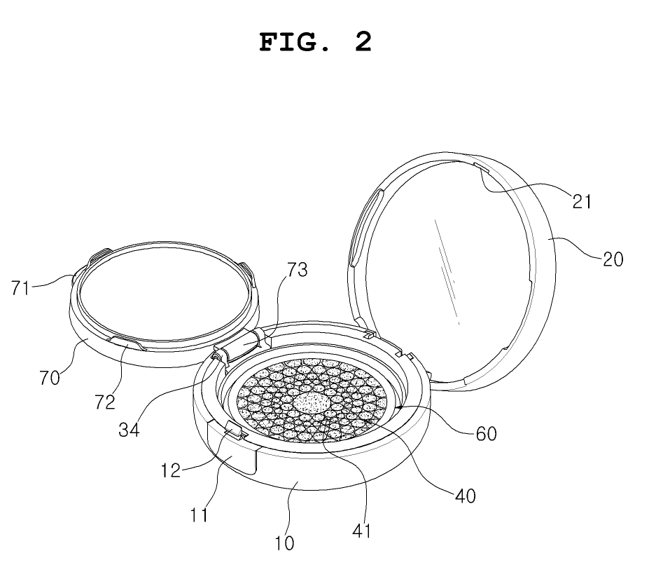

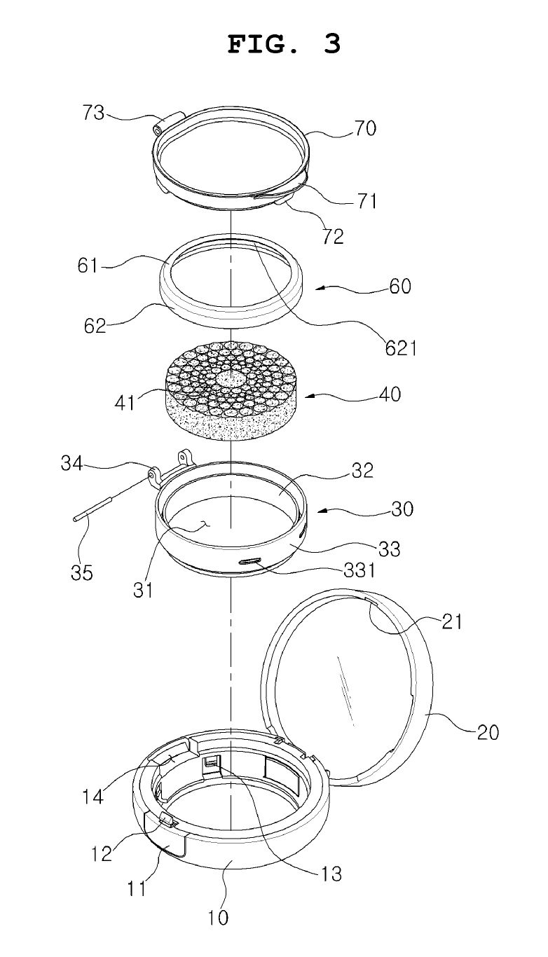

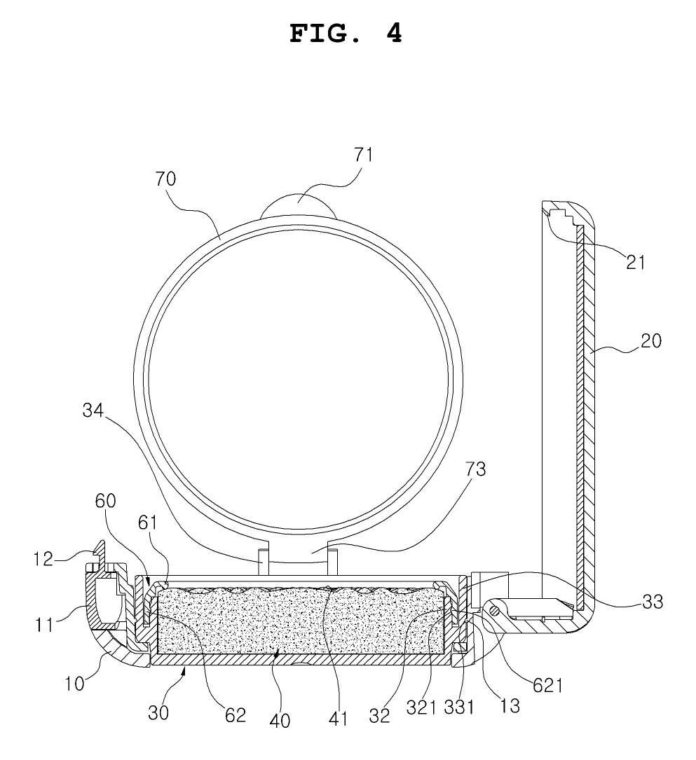

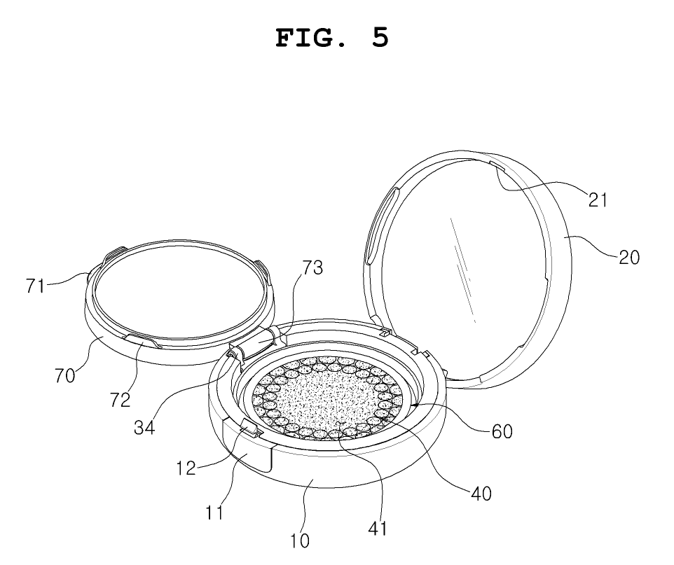

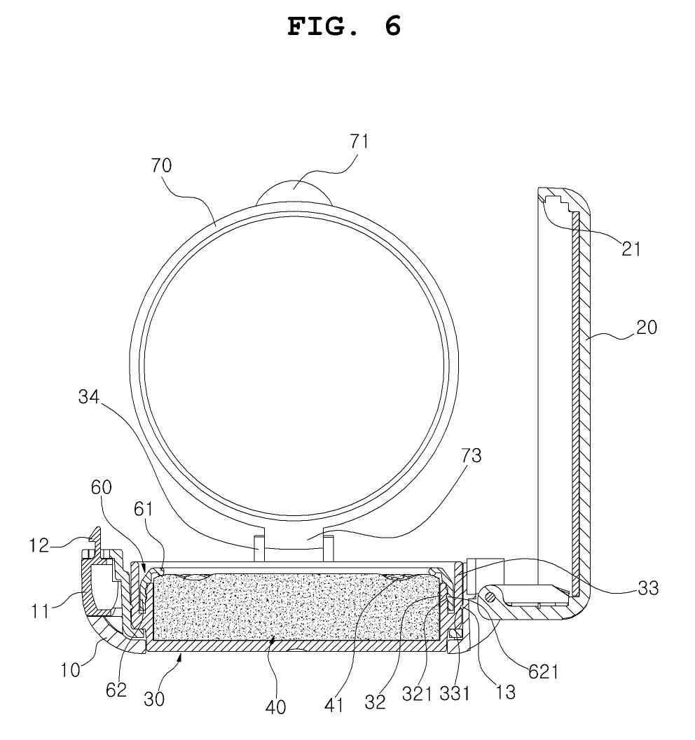

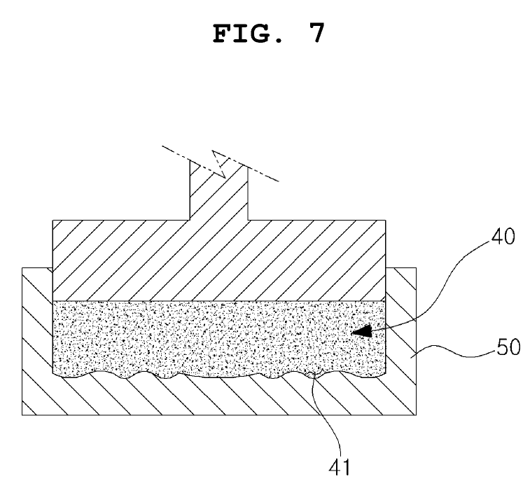

FIG. 2 is a perspective view showing a cosmetic product having an impregnation member embossed and engraved by melting a surface of the impregnation member according to an embodiment of the present invention, FIG. 3 is an exploded perspective view showing a cosmetic product having an impregnation member embossed and engraved by melting a surface of the impregnation member according to an embodiment of the present invention, FIG. 4 is a sectional view showing a cosmetic product having an impregnation member embossed and engraved by melting a surface of the impregnation member according to an embodiment of the present invention, FIG. 5 is a perspective view showing a cosmetic product having an impregnation member embossed and engraved by melting a surface of the impregnation member according to another embodiment of the present invention, FIG. 6 is a sectional view showing a cosmetic product having an impregnation member embossed and engraved by melting a surface of the impregnation member according to another embodiment of the present invention, and FIG. 7 is a sectional view showing a state of forming an impregnation member accommodated in a cosmetic product having the impregnation member embossed and engraved by melting a surface of the impregnation member according to an embodiment of the present invention.

According to a cosmetic product having an impregnation member embossed and engraved by melting a surface of the impregnation member of the present invention, the surface 41 of the impregnation member 40 is embossed and engraved by inserting and compressing the impregnation member 40 in a heated metal mold 50 formed with an emboss-engrave pattern and melting the surface 41, and the impregnation member 40 embossed and engraved by melting the surface 41 thereof is impregnated with a cosmetic material.

The cosmetic material impregnated in the impregnation member 40 may be a gel foundation containing sunscreen agents.

In addition, according to the present invention, the cosmetic product having the impregnation member embossed and engraved by melting the surface of the impregnation member includes: an outer container 10 having an open top; an outer container lid 20 coupled to one side of the outer container 10; an inner container 30 mounted inside the outer container 10; the impregnation member 40 mounted inside the inner container 30 and impregnated with a cosmetic material; and an inner container lid 70 hinged to one side of the inner container 30 so as to be opened and closed, wherein the surface 41 of the impregnation member 40 is embossed and engraved by inserting and compressing the impregnation member 40 in a heated metal mold 50 formed with an emboss-engrave pattern and melting the surface 41.

The outer container 10 is formed on one side surface thereof with a push button 11 having a locking sill 12, has a hinge formed on an opposite side of the push button 11 so as to be hinged to the outer container lid 20, is formed on an inner peripheral surface thereof with a fastening protrusion 13, and is formed on an inner periphery thereof with a hinge bracket mounting groove 14.

The push button 11 allows the locking sill 12 extending from an upper portion of the push button 11 to be easily pushed back by the pushing action of a user, so that the locking sill 12 can be separated from a locking protrusion 21 of the outer container lid 20.

The fastening protrusion 13 is fastened to a fastening groove 331 formed on an outer peripheral surface of an outer wall 33 of the inner container 30.

A hinge bracket 34 of the inner container 30 is inserted into the hinge bracket mounting groove 14.

The outer container lid 20 covers the upper portion of the outer container 10, and is hinged to the outer container 10 so as to be opened or closed.

The locking protrusion 21 is formed on one side of the outer container lid 20, and formed in a protruding shape to correspond to the locking sill 12 of the outer container 10.

The inner container 30 is mounted inside the outer container 10. In addition, the inner container 30 includes a bottom surface 31, an inner wall 32 extending upwards from the bottom surface 31, and an outer wall 33 spaced outwards from the inner wall 32 by a predetermined distance.

The inner wall 32 is formed on an outer peripheral surface thereof with a coupling protrusion 321, and the coupling protrusion 321 is fitted to a coupling groove 621 formed in a fixing member 60 to prevent the fixing member 60 from being separated from the inner container 30.

The outer wall 33 is formed on an outer peripheral surface thereof with a fastening groove 321, and the fastening groove 331 is fastened to the fastening protrusion 13 formed on the inner peripheral surface of the outer container 10 to prevent the inner container 30 from being separated from the outer container 10.

The outer wall 33 is formed on the outer peripheral surface thereof with the hinge bracket 34, and the hinge bracket 34 is hinged with a hinge block 73 formed on the inner container lid 70.

The impregnation member 40 is mounted inside the inner container 30 and impregnated with a cosmetic material, and the impregnation member 40 is formed of at least one material including butadiene rubber (BR), styrene butadiene rubber (SBR), natural rubber (NR), acrylonitrile-butadiene rubber (NBR), wet urethane, dry urethane, polyether, polyester, polyvinyl chloride, polyethylene, latex, silicone, polyvinyl alcohol (PVA), nitrile rubber, butyl rubber, and neoprene.

As shown in FIG. 7, the surface 41 of the impregnation member 40 is embossed and engraved by compressing the surface 41 in the heated metal mold 50 formed with the emboss-engrave pattern and melting the surface 41.

In the impregnation member 40 embossed and engraved on the surface 41 thereof, the open cell structure of the surface 41 is melted to convert the same into an open cell structure having an open cell that has a size of ½ or less, so that the cosmetic material can be thinly smeared on a puff so as to be efficiently used.

The impregnation member 40 is embossed on the surface 41 in various sizes of a convex circular or polygonal shape.

Since the open cell structure can be partially occluded when the surface 41 of the impregnation member 40 is melted with heat, the surface 41 is pressed with a plurality of needles to allow the occluded surface 41 to be perforated, so that the cosmetic material impregnated in the impregnation member 50 can be smoothly discharged.

In addition, as shown in FIGS. 5 and 6, according to another embodiment of the present invention, the impregnation member 40 can be embossed and engraved by melting only on the edge portion of the surface 41.

The fixing member 60 is coupled to the inner container 30, and the fixing member 60 includes a horizontal extension piece 61 extending inwards and a downward extension piece 62 extending downwards from the horizontal extension piece 61.

The horizontal extension piece 61 is seated at an upper end of the inner wall 32 of the inner container 30 to prevent the impregnation member 40 from being separated.

The downward extension piece 62 is formed on an inner peripheral surface thereof with the coupling groove 621, and the coupling groove 621 is coupled to the coupling protrusion 321 formed on an outer peripheral surface of the inner wall 32 of the inner container 30 to prevent the fixing member 60 from being separated from the inner container 30.

The inner container lid 70 is coupled to one side of the inner container 30 so as to open and close the inner container 30.

The inner container lid 70 is formed on one side thereof with a handle 71, formed on a lower end thereof with a sealing piece 72, and has the hinge block 73 formed on an opposite side of the handle 71.

The handle 71 makes it easier to open and close the inner container lid 70.

The sealing piece 72 is fitted inside the outer wall 33 of the inner container 30 to improve sealing performance of the inner container 30 when the inner container lid 70 is closely covered on the inner container 30.

The hinge block 73 is fitted to the hinge bracket 34 of the inner container 30, and is fixed by a hinge pin 35.

Hereinafter, a method of assembling and using the cosmetic product having the impregnation member embossed and engraved by melting the surface of the impregnation member according to an embodiment of the present invention will be described in detail.

In order to assemble the cosmetic product having the impregnation member embossed and engraved by melting the surface of the impregnation member of the present invention, the outer container lid 20 is coupled to the outer container 10 on which the push button 11 is formed.

Then, the inner container lid 70 is coupled to the inner container 30.

The impregnation member 40 impregnated with the cosmetic material is mounted in the inner container 30 coupled with the inner container lid 70, in which the surface 41 of the impregnation member 40 is embossed and engraved by compressing the surface 41 in the heated metal mold 50 formed with the emboss-engrave pattern and melting the surface 41 as shown in FIG. 7.

The impregnation member 40 is embossed on the surface 41 in various sizes of a convex circular or polygonal shape.

In addition, as shown in FIGS. 5 and 6, according to another embodiment of the present invention, the impregnation member 40 can be embossed and engraved by melting only on the edge portion of the surface 41.

Thereafter, the fixing member 60 is coupled to the inner container 30 in which the impregnation member 40 is mounted, and the inner container 30 is coupled inside the outer container 10 to complete the assembly.

In the impregnation member 40 of the cosmetic product having the impregnation member embossed and engraved by melting the surface of the impregnation member, which is assembled as described above, the open cell structure of the surface 41 is melted to convert the same into an open cell structure having an open cell that has a size of ½ or less, so that a proper amount of the cosmetic material can be uniformly and thinly smeared on the puff, so that the cosmetic material can be efficiently used.

The impregnation member 40 is embossed on the surface 41 in various sizes of a convex circular or polygonal shape, so that various designs can be implemented to broaden the range of a consumer's choice.

As described above, although the cosmetic product having the impregnation member embossed and engraved by melting the surface of the impregnation member according to one embodiment of the present invention has been described for illustrative purposes, the present invention is not limited thereto. It is understood that various changes and modifications can be made by those skilled in the art without departing from the spirit and scope of the present invention as disclosed in the accompanying claims.

[Description of Reference numerals]

| 10: Outer container | 11: Push button |
|---|---|
| 12: Locking sill | 13: Fastening protrusion |
| 14: Hinge bracket mounting groove | |
| 20: Outer container lid | 21: Locking protrusion |
| 30: Inner container | 31: Bottom surface |
| 32: Inner wall | 321: Coupling protrusion |
| 33: Outer wall | 331: Fastening groove |
| 34: Hinge bracket | 35: Hinge pin |
| 40: Impregnation member | 41: Surface |
| 50: Metal mold | 60: Fixing member |
| 61: Horizontal extension piece | |
| 62: Downward extension piece | |
| 621: Coupling groove | 70: Inner container lid |
| 71: Handle | 72: Sealing piece |

73: Hinge block

1. A cosmetic product having an impregnation member embossed and engraved by melting a surface of the impregnation member, wherein the surface (41) of the impregnation member (40) is embossed and engraved by inserting and compressing the impregnation member (40) in a heated metal mold (50) formed with an emboss-engrave pattern and melting the surface (41), and the impregnation member (40) embossed and engraved by melting the surface (41) thereof is impregnated with a cosmetic material.

2. A cosmetic product having an impregnation member embossed and engraved by melting a surface of the impregnation member, the cosmetic product comprising:
an outer container (10) having an open top;
an outer container lid (20) coupled to one side of the outer container (10);
an inner container (30) mounted inside the outer container (10);
the impregnation member (40) mounted inside the inner container (30) and impregnated with a cosmetic material; and
an inner container lid (70) hinged to one side of the inner container (30) so as to be opened and closed,
wherein the surface (41) of the impregnation member (40) is embossed and engraved by inserting and compressing the impregnation member (40) in a heated metal mold (50) formed with an emboss-engrave pattern and melting the surface (41).

3. A cosmetic product having an impregnation member embossed and engraved by melting a surface of the impregnation member, the cosmetic product comprising:
an inner container (30);
the impregnation member (40) mounted inside the inner container (30) and impregnated with a cosmetic material; and
an inner container lid (70) hinged to one side of the inner container (30) so as to be opened and closed,
wherein the surface (41) of the impregnation member (40) is embossed and engraved by inserting and compressing the impregnation member (40) in a heated metal mold (50) formed with an emboss-engrave pattern and melting the surface (41).

4. The cosmetic product of claim 2, wherein a fixing member (60) is further coupled to an upper end of the inner container (30) to fix the impregnation member (40) such that the impregnation member (40) is prevented from being separated to an outside.

5. The cosmetic product of claim 1, wherein the surface (41) of the impregnation member (40) is melted and occluded by the metal mold (50), and the occluded surface (41) is perforated by a plurality of needles.

6. The cosmetic product of claim 1, wherein the impregnation member (40) is embossed and engraved only on an edge portion of the surface (41).

7. The cosmetic product of claim 1, wherein the impregnation member (40) is embossed on the surface (41) in a convex circular or polygonal shape.

8. The cosmetic product of claim 3, wherein a fixing member (60) is further coupled to an upper end of the inner container (30) to fix the impregnation member (40) such that the impregnation member (40) is prevented from being separated to an outside.

9. The cosmetic product of claim 2, wherein the surface (41) of the impregnation member (40) is melted and occluded by the metal mold (50), and the occluded surface (41) is perforated by a plurality of needles.

10. The cosmetic product of claim 3, wherein the surface (41) of the impregnation member (40) is melted and occluded by the metal mold (50), and the occluded surface (41) is perforated by a plurality of needles.

11. The cosmetic product of claim 2, wherein the impregnation member (40) is embossed and engraved only on an edge portion of the surface (41).

12. The cosmetic product of claim 3, wherein the impregnation member (40) is embossed and engraved only on an edge portion of the surface (41).

13. The cosmetic product of claim 2, wherein the impregnation member (40) is embossed on the surface (41) in a convex circular or polygonal shape.

14. The cosmetic product of claim 3, wherein the impregnation member (40) is embossed on the surface (41) in a convex circular or polygonal shape.
